(19) United States
(12) Patent Application Publication
(10) Pub. No.: US 2025/0186490 A1
(43) Pub. Date: Jun. 12, 2025

(54) METHOD OF CHEMICALLY TREATING OR CULTIVATING MACROPHAGES AND THEIR THERAPEUTIC APPLICATIONS IN FIBROTIC DISEASES

(71) Applicant: MAPONOS THERAPEUTICS INC., Mount Prospect, IL (US)

(72) Inventors: Xiaoyang WU, Chicago, IL (US); Yingming ZHAO, Chicago, IL (US)

(21) Appl. No.: 18/843,132

(22) PCT Filed: Mar. 7, 2023
(86) PCT No.: PCT/US2023/014702

§ 371 Date: Aug. 30, 2024

Related U.S. Application Data
(60) Provisional application No. 63/317,291, filed on Mar. 7, 2022.

(51) Int. Cl. A61K 35/15; A61P 1/16; A61P 9/00; A61P 11/00; A61P 13/12; C12N 5/0786

(52) U.S. Cl. CPC A61K 35/15; A61P 1/16; A61P 9/00; A61P 11/00; A61P 13/12; C12N 5/0645; C12N 2500/02; C12N 2533/30

(57) ABSTRACT

A novel cellular product composed of chemically-treated macrophages for treating fibrosis diseases. The cellular product has the beneficial effect of protecting organs against fibrosis-induced lesions and dysfunction by reducing collagen content. The macrophages are treated with mitochondrial respiratory chain inhibitors and cultured under hypoxia conditions. They express a higher level of anti-fibrosis bio-markers and a reduced level of pro-fibrosis bio-markers. The macrophages can be administered to animals with pulmonary fibrosis via tail vein or intratracheal injection. The invention can also be used to treat patients with Idiopathic Pulmonary Fibrosis, liver fibrosis, myocardial fibrosis, and renal fibrosis. A novel technique produces and manufactures cellular products for therapeutic purposes by using tissue culture vehicles coated with temperature-responsive polymer material. This novel technique has a clear advantage in growing more cells and preserving cell integrity compared to conventional methods.

This application claims the priority of U.S. Ser. No. 63/317,291, filed Mar. 7, 2022. The entire contents and disclosure of the preceding application are incorporated by reference into this application.

### FIELD OF THE INVENTION

The present invention relates to chemically-treated Macrophages and their therapeutic applications in fibrotic diseases. This invention also relates to the cultivation of cells such as macrophages and their use as therapeutic products.

### BACKGROUND OF THE INVENTION

Fibrosis is a pathological process defined by the excessive accumulation of collagen and other extracellular matrix (ECM) in an organ that can lead to the distortion of normal tissue architecture and the impairment of tissue functions. Fibrosis can be caused by trauma, medical injury and disease, and it is the outcome of dysfunctional tissue repair responses in many types of tissue damage especially during chronic inflammatory diseases.

When tissue is injured, local fibroblast activation, secretion of inflammatory mediators, and synthesis of extracellular matrix (ECM) (e.g., collagen and fibronectin) occur, and these changes combine to initiate the healing response. Under normal circumstances, the physiological repair process has two stages: the first is regeneration, in which new cells of the same type replace cells of the damaged tissue, and the second stage involves fibrotic proliferation, in which connective tissue replaces normal tissue. However, when the injury is severe or recurrent, the repair process is uncontrolled, it can lead to massive deposition of ECM, which can turn normal tissue into permanent tissue scarring. The ECM components continue to accumulate, leading to structural damage, organ dysfunction and even failure.

Fibrosis can occur in almost all organs in the body, thus damaging the structure and function of the tissue. Common diseases associated with fibrosis include cirrhosis, hepatitis, non-alcoholic steatohepatitis (NASH), chronic kidney disease, myocardial infarction, heart failure, diabetes, idiopathic pulmonary fibrosis (IPF) and scleroderma. Globally, fibrosis has been one of the leading causes of disability and death in many diseases, to the extent that fibrosis causes 45% of all deaths in the industrialized world and affects nearly a quarter of the population worldwide, imposing a heavy burden on health care systems and individuals.

US Patent Pub. No. 20210100837A1 described a novel assay of genetically-engineered macrophages to treat fibrosis and reduce fibrotic lesions in multiple organs, including liver, lung, and heart. The macrophages can be genetically engineered to express a recombinant targeting protein and/or a recombinant catalytic enzyme. US Patent Pub. No. 20200405757A1 described an assay that can use unpolarized monocyte-derived macrophages to treat liver cirrhosis. The publication describes the method of isolating and culturing autologous macrophages from human patients. The publication shows the characterization of said macrophages and the evaluation of their anti-fibrosis effect in patients with liver cirrhosis. The current invention disclosed herein is the first to report that chemically-treated microphases can increase the anti-fibrosis capacity of the cells. The novelty of this invention lies in the steps of the chemical treatment method. There is no previous publication report of such an innovative method which can increase the anti-fibrosis effect of macrophages. The essential innovative steps of this invention involve the experimental protocol of treating the macrophages with certain chemicals then culturing them under specific conditions.

There are two prior publications as referenced above, both of which describe a method using macrophages to treat fibrosis diseases. One uses autologous unpolarized macrophages to treat liver cirrhosis; the other uses genetically-engineered macrophages to treat fibrosis in multiple animal models. Compared to the said patented methods, this current invention uses a specific type of chemicals, Mitochondrial Respiratory Chain inhibitors (MRCi), to treat the macrophages, then the treated macrophages are subjected to a very specific culture condition in order to achieve the enhanced anti-fibrosis capacity. Since the treatment process and the culture condition are both very specific and not considered regular cell culture processes, therefore the current invention cannot be discovered or led to through obvious or regular cellular experiment practice based on the two prior arts.

Macrophages as important immune cells, are widely distributed in many tissues of the body and have a very important role in maintaining the homeostasis of the body's internal environment, providing for the maintenance of homeostasis and tissue defense in the body, being the first line of defense of the body's immune response, and playing an important role in both the innate and acquired immune responses, and their phagocytic capacity is one of the markers of the body's nonspecific immune function.

Macrophages are a heterogeneous group of cells involved in maintaining tissue homeostasis, removing apoptotic cells and cellular debris to aid in tissue remodeling and repair. The clearance process is an important metabolic contribution without which the host would not survive; Macrophages are also involved in the removal of cellular debris generated during tissue remodeling and in the rapid and efficient removal of cells that have undergone apoptosis. In some cases, macrophages can enter a pro-inflammatory state and act mainly as a defense against external pathogens or participate in the repair of damaged tissues. Therefore, primary extraction and culture of macrophages are important for human immunology, immune injury and molecular cell biology.

In addition to phagocytosis, macrophages have chemotactic, secretory, and antigen-presenting functions and have roles in all aspects of organism biology, with multifunctional roles and high plasticity, making them the subject of research in many fields. There are previous studies that show that macrophages can be a powerful tool for gene and cell therapy for a wide range of conditions. Among others, macrophages have been shown to have a very high potential in the development of new therapies for cancer and fibrotic diseases.

There are still many technical hurdles in the development of cell therapy therapies around macrophages. Macrophages exist in most tissues of the body, and macrophages in tissues are extremely heterogeneous and belong to a non-reproducing cell population, which is difficult to survive for a long time, and it is difficult to extract and enrich them, so it is imperative to establish a system for extracting and culturing macrophages.

Another major challenge in the culture of macrophages is the selection of culture vessels and detachment reagents. The nature of macrophages growth results in that they are not easy to be harvested, while conventional culture methods and trypsin use not only lose a large number of cells during cell harvesting process but also tend to affect the state of the cells themselves.

U.S. Pat. No. 5,284,766 described a novel bed material for cell culture which coats the tissue culture polystyrene (TCPS) with Poly (N-isoproplyacrylamide) (PIPAAm). Cells cultured in Petri dishes coated with PIPAAm were detached when the temperature dropped below 30° C. In comparison to the conventional cell culture dishes without PIPAAm coating and used enzyme-based detachment agents, cells harvested using temperature-responsive methods showed a higher growth rate.

The present invention takes a novel approach to utilize a temperature-responsive material for macrophage culturing, which has the surface of the TCPS dishes spread with PIPAAm and its copolymer. PIPAAm is considered as an intelligent polymer that has reversible temperature-responsive soluble/insoluble character changes in aqueous solution below and above a lower critical solution temperature (LCST) of 32° C. Culturing macrophages on the PIPAAm-coated TCPS avoids the destruction of ECM, cellular junctions and cell membrane proteins such as ion channels and growth factor receptors caused by conventional chelating agents such as EDTA or proteolytic enzymes such as trypsin. This invention solves the problem that macrophages are difficult to harvest, and improves the cell viability in the process, while maximizing the preservation of cell status.

### SUMMARY OF THE INVENTION

Aiming at developing an effective and safe approach in treating fibrosis diseases, the present invention provides a novel cellular product having the beneficial effect of protecting organs against fibrosis-induced lesions and dysfunction, wherein the cellular product is composed of chemically-treated macrophages. This cellular product has the capacity of reducing collagen contents and is convenient to be administrated and potentially is generally applicable for treating fibrosis in multiple organs.

In one aspect of this intervention, chemically-treated macrophages can express a higher level of anti-fibrosis bio-markers. In certain aspects, chemically-treated macrophages also express a reduced level of pro-fibrosis bio-markers. In certain aspects of the embodiments, macrophages can be treated to reduce fibrosis contents in an animal model with pulmonary fibrosis.

In one embodiment, macrophages isolated from the bone marrow of mice for use in accordance with any aspect or embodiment of the invention can be cultured in RPMI-1640 medium with the presence of Macrophage Colony-Stimulating Factor (M-CSF). By the sixth day of the culturing period, cells can be treated with mitochondrial respiratory chain inhibitors (MRCi) like Rotenone, Deguelin, Capsaicin, or Piericidin, and subsequently be cultured under hypoxia conditions. Chemically-treated macrophages are characterized by the expression of higher anti-fibrosis marker CXCL-10, and lower expression of pro-fibrosis marker MCP-1.

In one embodiment, macrophages can be chemically treated with MRCi and then cultured under hypoxia conditions for treating both silica- and bleomycin-induced pulmonary fibrosis. By the end of the culture process, macrophages can be collected and then administrated into mice with silica- and bleomycin-induced pulmonary fibrosis either via tail vein injection or via direct injection intratracheally. In some embodiments, hydroxyproline is measured to evaluate the fibrotic content in the lung tissues. Mice with pulmonary fibrosis received said macrophages show a reduction in hydroxyproline content compared to mice who received unpolarized, un-treated macrophages.

In one embodiment of the invention, patients with fibrosis diseases may be treated by administration of said macrophages. Autologous isolated unpolarized human macrophages can be chemically treated and then administered to patients diagnosed with Idiopathic Pulmonary Fibrosis or IPF by the end of the culture and treatment process. In a further aspect, said chemically-treated macrophages can also be used to treat patients with liver fibrosis or cirrhosis. In other aspects, said macrophages can also be used to treat patients with myocardial fibrosis caused either by acute heart injuries like myocardial ischemia or by chronic heart diseases, including hypertension, heart failure, and diabetic cardiomyopathy. In some aspects of this intervention, said macrophages can also be applied in clinical practice for treating patients with renal fibrosis caused by chronic kidney diseases (CKD).

The current invention disclosed herein is the first to report that growing macrophages in PIPAAm-coated TCPS improves cell viability during the culture harvesting process. The novelty of this invention is evidenced by the fact that no previous report shows the usage of this particular cell culture material for the growth and harvesting of macrophages for cell therapeutic purposes using macrophages. The critical component of this present invention comprises the macrophage culture method using a Nunc™ Dishes with UpCell™ Surface, which is manufactured by and commercially available through Thermo Fisher Scientific. There is a Prior Art as listed above that describes a method using PIPAAm-coated TCPS to culture bovine aorta endothelial cells and focused on the temperature-responsive property of the PIPAAm. Instead, present invention uses the PIPAAm for culturing and harvesting of macrophages. What is further differentiate from the previous art is that the macrophages applied in present invention is set for the therapeutic applications and future clinical treatments for various diseases.

The present invention describes a novel technique in producing and manufacturing cellular products for therapeutic purposes, wherein the method is to use a specific temperature-responsive polymer material for coating of tissue culture vehicles, namely Poly (N-isoproplyacrylamide) (PIPAAm) and its copolymer. The said novel technique demonstrates a clear advantage in terms of growing more cells and preserving cell integrity, evidenced by higher cell number and viability, in comparison to conventional cell culture and harvesting methods.

The growing and harvesting of macrophages have long been a challenge in related studies and are considered one of the bottleneck issues when it comes to large-scale cell manufacturing for potential experimental and clinical practices due to the stronger adhesion of the cells to conventional culture TCPS. To harvest the macrophages, classic techniques include mechanically scrapping of the surface cell, using enzyme-based dissociation agents like trypsin-EDTA, and using enzyme-free agents to preserve the surface protein. Yet all these methods suffer from low cell viability and loss of cell yields.

In 1990, Masayuki Yamato and colleagues developed a novel TCPS using temperature-responsive polymers PIPAAm. The polymer-coating transits from hydrophobic to hydrophilic in response to an environmental temperature lower or higher than a critical solution temperature (32° C.). When the TCPS is placed in a normal tissue culture temperature (37° C.), the hydrophobic coating surface allows the cells to attach and grow to confluence. When placed at a temperature below 30° C., i.e., ambient room temperature, the surface becomes hydrophilic and expels the cell to float without damaging the cell surface protein.

In one embodiment, CD14+ monocytes can be isolated and differentiated from human PBMC for use in accordance with any aspect or embodiment of the invention. At the end of the isolation process, CD14+ monocytes can be seeded in either PIPAAm coated TCPS or traditional TCPS without the temperature-responsive coating and cultured in TexMACs medium with the presence of Macrophage Colony-Stimulating Factor (M-CSF). By the seventh day of the culturing period, cells from both types of TCPS can be subjected to multiple harvesting methods.

In one embodiment, macrophages grown on the PIPAAm-coated TCPS can be harvested through temperature changes from 37° C. to ambient room temperature, while macrophages grown on TCPS without PIPAAm coating can be harvested either through mechanically scrapping with cell lifters or applying a commercially available enzyme-free Cell Dissociation Buffer. All cells harvested through different methods are subjected to cell viability tests and the cell yields can be counted as well.

### BRIEF DESCRIPTION OF DRAWINGS

FIGS. 1A-1E show compound-treated macrophages exhibited enhanced anti-fibrosis ability in bone marrow-derived macrophages in vitro. FIGS. 1A and B shows the ELISA testing result of CXCL 10, in which Bone Marrow-Derived Macrophages (BMDM) treated with 1 μM rotenone or 10 μM deguelin both had an increased CXCL 10 level. CXCL 10 is considered as a protective factor against fibrosis development in pulmonary fibrosis by inhibiting fibroblast migration.

FIGS. 1C, 1D, 1E show the MCP-1 level in both compound-treated and untreated BMDMs. ELISA analysis illustrated treating BMDM with MRCi. In these cases, 1 μM rotenone, 5 μM capsaicin, or 0.1 μM Piericidin A all could individually reduce the MCP-1 level in BMDMs. MCP-1 is the abbreviation of monocyte chemoattractant protein 1, which is a type of chemokine that upregulates in inflammatory and fibrotic processes. A decrease in MCP-1 level can be considered as an enhanced anti-fibrosis indicator. Combining these two results, we can conclude that treating with MRCis like Rotenone and then culturing under hypoxia conditions can significantly increase the anti-fibrosis effect of BMDM.

FIGS. 2A and 2B demonstrate the chemically-treated macrophages were endowed with stronger anti-fibrosis capacity in vivo. Both figures show the results of the biochemical analysis of hydroxyproline. Hydroxyproline being a major component of tissue collagen, is often used as the most recognized indicator of ECM deposition and severity of fibrosis diseases. N: Normoxia; H: Hypoxia; R: Rotenone.

FIG. 2A shows macrophages delivered via tail injection reduced the hydroxyproline content in mice, and macrophages treated with Rotenone and then cultured under hypoxia condition had a greater hydroxyproline reduction compared to the untreated cells. Yet, treating BMDMs with rotenone and then culturing them under a normal oxygen environment did not demonstrate the same enhanced anti-fibrotic capacity. FIG. 2B demonstrated a similar result in mice that received macrophages through direct intratracheal delivery. These figures demonstrated that macrophages that had been treated with certain MRCi and then cultured under hypoxia conditions could further reduce the collagen contents in mice with pulmonary fibrosis more effectively than the macrophages without chemical compound treatment.

FIG. 3 shows the cell growth result from three groups of samples. Macrophages that grew in the UpCell dishes had the highest cell numbers in comparison to the cells that grew in regular culture dishes and were harvested using a cell lifer or Cell Dissociation Buffer. This result demonstrated culture dishes coated with PIPAAm had enhanced capacity of cell yields.

FIG. 4 shows the viability of cells harvested from both groups. In the UpCell group, cells harvested through temperature changes had a 95.92±2.36% viability, while cells harvested through gentle scraper with cell lifters or the Cell Dissociation Buffer only had 82.48±3.88% and 76.61±9.57% viability, respectively.

FIG. 5 shows the hydroxyproline contents, wherein “3M Cells” refers to 3 million macrophages injected into mice.

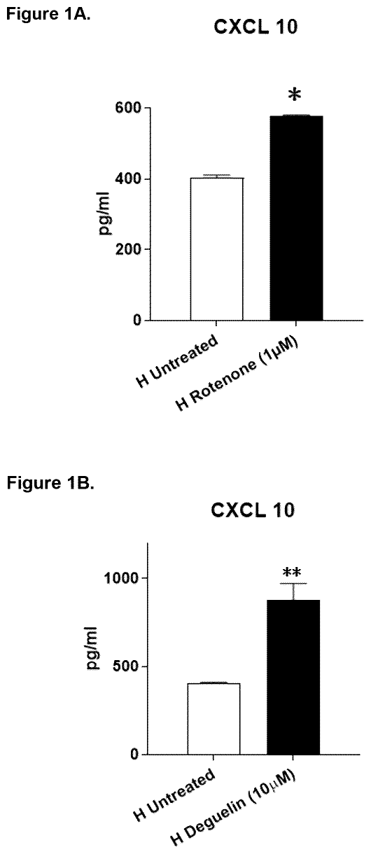

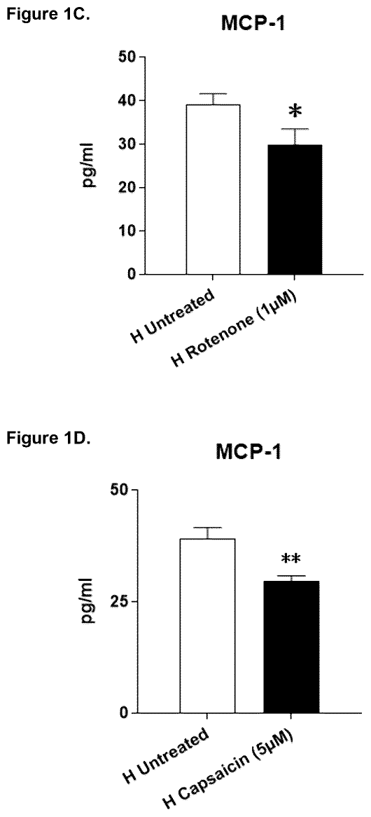

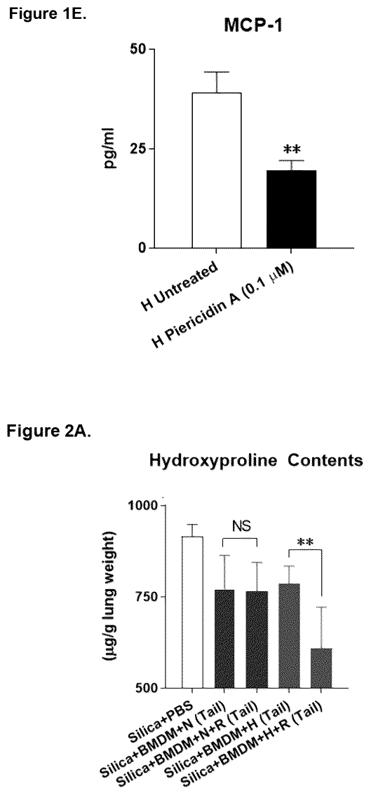

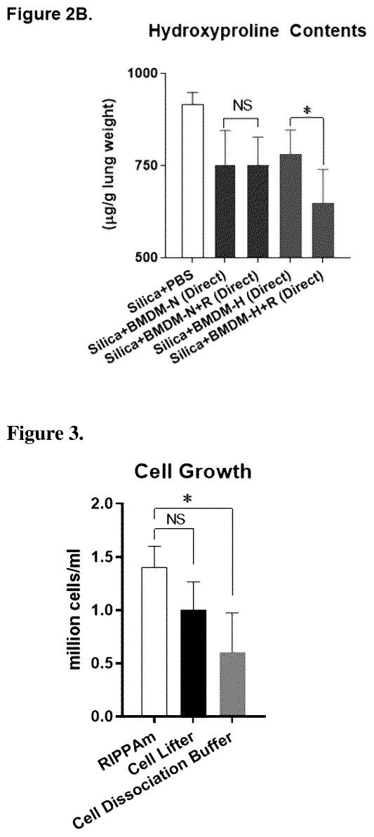

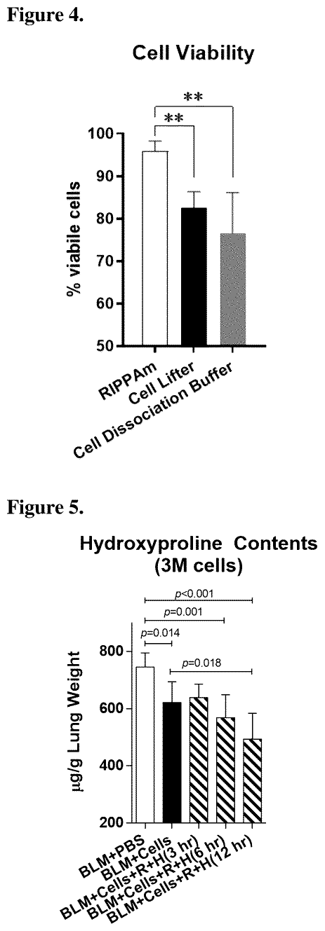

### DETAILED DESCRIPTION OF THE INVENTION

Embodiments described herein are directed to chemically-treated macrophages with enhanced anti-fibrosis capacity in pulmonary fibrosis and other organ fibrosis. This disclosure is further directed to a series of cellular therapy products based on autologous isolated macrophages. Further, this invention is directed to novel therapeutic approaches to reduce fibrotic contents and reconstruct normal tissues in the lung, liver, heart, kidney, and other organs with fibrosis by administering chemically-treated macrophages.

It was hypothesized that glycolysis and lactate production might promote the anti-fibrotic effect in BMDM, and MRC was involved in the pro-inflammatory response of the BMDM. It was subsequently observed that MRCi-treated BMDM enhanced BMDM's anti-fibrosis ability under hypoxic conditions. The in vitro ELISA data and the in vivo results supported the conclusion that BMDM itself possessed a certain degree of anti-fibrotic qualities. Treating BMDM under normal culture conditions did not change the degree of fibrosis reduction. Treating BMDM with MRCi and then culturing them under hypoxia condition did enhance the anti-fibrosis capacity by 15% to 20%. Yet detailed and exact mechanism of action behind this phenomenon is still unclear.

The following terms shall be used to describe the present invention. In the absence of a specific definition set forth herein, the terms used to describe the present invention shall be given their common meaning as understood by those of ordinary skill in the art.

Macrophages are white blood cells located in tissues and are derived from monocytes, which in turn are derived from precursor cells in the bone marrow. Both macrophages and monocytes are phagocytes that participate in innate immunity and cellular immunity. Together with neutrophils, they are the first responders to infection. Macrophages are involved in the recognition, phagocytosis and degradation of cellular debris and pathogens in the form of fixed or free cells and to activate lymphocytes or other immune cells to speed up their response time to pathogens. Macrophages also play a role in presenting antigens to T cells and in inducing the expression of costimulatory molecules by other antigen-presenting cells, thus initiating an adaptive immune response. In addition, they play an important role in the early stages of inflammation through the release of cytokines and chemokines that in turn recruit other immune cells to the site of inflammation.

Macrophages are present in most tissues and therefore have different functions. In addition to initiating immune and inflammatory responses to pathogens, macrophages also play a role in maintaining tissue homeostasis and in tissue repair and remodeling. Unfortunately, this function has been associated with many diseases, including metabolic and autoimmune diseases, cancer, infection, obesity, and fibrosis. Thus, macrophages also seem to play a key role in the tumor microenvironment, particularly in stromal remodeling, angiogenesis, metastasis, and tumor progression.

During the conventional immune response, pro-inflammatory macrophages are suppressed, resulting in a reduction of their pro-inflammatory signaling. During prolonged injury, dysregulated macrophages continue to secrete inflammatory cytokines and recruit other immune cells. These processes maintain chronic inflammation and are thought to play an important role in tumorigenesis and development. Once a tumor forms, it causes macrophages to differentiate from an immune active state to an immune-suppressed state. During wound healing, macrophages may also lead to fibrosis if the immune response is not sufficiently controlled. For other chronic diseases, including atherosclerosis, asthma, inflammatory bowel disease and rheumatoid arthritis, macrophages also play a key role.

Macrophages can be classified into classically activated macrophages (M1 type) and selectively activated macrophages (M2 type). M1 macrophages are involved in pro-inflammatory responses and play a central role in host defense against bacterial and viral infections. M1 type macrophages are activated by lipopolysaccharide (LPS) and interferon γ (IFN-γ) and upon activation M1 type macrophages secrete a large number of cytokines, such as NO, tumor necrosis factor α (TNF-α) and interleukin 6 (IL-6), which have pro-inflammatory and bactericidal effects. M2 type polarization mainly leads to anti-inflammatory, tissue repair, vascular regenerative and pro-tumor effects, and can be divided into three subtypes: a, b and c. M2a is activated by IL-4 and IL-13; M2b is activated by immune complex (IC) and Toll-like receptors (TLRs); and M2c is activated by IL-10 and glucocorticoids. Whereas the classification of the above subtypes may not fully represent the complexity of the transition state during macrophage activation, macrophage typing is closely related to changes in the microenvironment. In addition, researchers have identified other classes of macrophages, such as CD169+ macrophages and T-cell antigen receptor T cells (TCR+) macrophages.

Applied in the present invention are “unpolarized macrophages”, which are macrophage progenitors or precursor cells that have been fully differentiated from source cells, such as monocytes, but have not yet been polarized. In some embodiments of the present invention, unpolarized macrophages are monocyte-derived cells. Unpolarized macrophages can be obtained from, for example, CD14-positive monocytes from a bone marrow sample, followed by isolation and differentiation of monocytes to obtain unpolarized macrophages.

In some embodiments, unpolarized macrophages are generated from bone marrow cell-derived monocytes from mice according to the culture methods and conditions described in the example section herein. Bone marrow from murine tibia and femur can be flushed out with PBS then go through a red blood cell lysis step to remove the red blood cells in the bone marrow cells. Bone marrow cells then can be cultured in a regular RPMI1640 medium with the presence of Macrophage Colony-Stimulating Factor (M-CSF, R&D Systems, MN) at a concentration of 100 ng/ml. On day six, cells can be treated with mitochondrial respiratory chain inhibitors (MRCi) for two hours and cultured under hypoxia conditions for one additional day as described below in Example 1.

Throughout this application, various references or publications are cited. Disclosures of these references or publications in their entireties are hereby incorporated by reference into the application in order to more fully describe the state of the art to which this invention pertains. It is to be noted that the transitional term “comprising”, which is synonymous with “including”, “containing” or “characterized by”, is inclusive or open-ended, and does not exclude additional, un-recited elements or method steps.

The present invention will be better understood by reference to the examples as follows, but those skilled in the art will readily appreciate that the specific examples detailed are only for illustrative purposes and are not meant to limit the present invention as described herein, which is defined by the claims following thereafter.

### Example 1: Enhanced Anti-Fibrotic Effect of Chemically Treated Macrophages In Vitro

### I. Macrophages Isolation, Culturing, and Chemical Treatment.

In this example, macrophages were isolated and cultivated from the source of murine bone marrow. Eight-week-old male C57/BL6 mice (The Jackson Laboratory, ME, USA) were sacrificed by cervical dislocation. The tibia and femur were separated, and the bone marrow was flushed out using ice-cold PBS solution. Bone marrow cells were then resuspended into single-cell suspension and washed with ice-cold PBS. The cells were treated with Red Blood Cell Lysis Buffer (MilliporeSigma, MA, USA). After the treatment, the cell suspension was washed with ice-cold PBS then seeded with a culture medium.

The culture medium for the BMDM used in this example includes RPMI1640 (Thermo Fisher Scientific, MA, USA) containing 10% heat-inactivated Fetal Bovine Serum (Gemini Bio, CA, USA), supplied with 100 ng/ml of Recombinant Mouse M-CSF Protein (R&D Systems, MN, USA). Fresh medium was supplied on day three and day five post isolation. On day six of the culturing process, 1 μM of Rotenone (MilliporeSigma, MA, USA) in DMSO solution was added into the culturing medium and incubated for 2 hours with the cells. Then the culture was replaced with fresh medium and was placed into a hypoxia culture chamber with 1% oxygen supply and 5% carbon dioxide at 37° C. On day seven, the cells were ready for downstream treatments. In the untreated group, only vehicle (DMSO) was added as the control. In addition to Rotenone, the following mitochondrial respiratory chain inhibitors (MRCi) were also tested following the same culturing process: Deguclin (10 μM), Piericidin A (0.1 μM), and Capsaicin (5 μM).

II. In Vitro Testing of the Enhanced Anti-Fibrotic Effect of the Chemical Compound-Treated Macrophages with ELISA Assays.

To evaluate the enhanced anti-fibrosis ability of the chemically-treated BMDM, ELISA assays were applied using commercially purchased testing kits. In this example, a Mouse MCP-1 ELISA Kit and a Mouse IP-10 ELISA Kit (CXCL10) (both from Abcam plc, Cambridge, UK) were used. Tests using both kits were performed according to the instructions provided by Abcam. Briefly, supernatants of the cell culture were removed from the culture plates and centrifuged to remove the debris, then diluted supernatants were added to the microplate stripes. Prepared antibody cocktails were then mixed with the culture supernatants and incubated according to the instruction. By the end of the incubation period, after adding the development reagent and the stop reagent, the microplates were placed into a spectrometer to record the set OD.

The results of this study show that, in comparison to the untreated cells, the chemically treated macrophages demonstrated enhanced anti-fibrotic ability evidenced by the increased CXCL10, an anti-fibrosis marker, and reduced MCP-1, which is a pro-fibrosis marker.

### Example 2: Reduction of Fibrosis in a Murine Pulmonary Fibrosis Model with Chemically-Treated Macrophages

### 1. Establishing a Murine Pulmonary Fibrosis Model.

In this example, a silica-induced murine pulmonary fibrosis model was established to evaluate the anti-fibrosis ability of the chemically-treated macrophages. The silica-induced fibrosis model is one of the most widely used animal models to study pulmonary fibrosis diseases. Eight-week-old male C57/BL6 mice were used in this study. Mice were anesthetized with isoflurane inhalation, then exposed to 10 mg silica (MilliporeSigma, MA, USA) dissolved in 40 μl of PBS.

### II. Engraftment of Chemically-Treated Macrophages and Biochemical Analysis.

Three weeks post the initial silica exposure, BMDM were isolated, treated with Rotenone and then cultured under both normoxia and hypoxia conditions as described in Example 1. On day seven of the culturing process, both compound-treated and untreated macrophages were administrated into mice exposed to silica cither via tail vein injections or direct intratracheal delivery. BMDM cultured under normoxia conditions were administrated as controls. Mice were sacrificed seven days post the macrophages engraftments. Lung tissues were collected for subsequent tests.

A hydroxyproline Colorimetric Assay Kit (Bio Vision Inc., CA, USA) was used to examine the collagen contents in the murine lung tissues. Tissues were homogenized and hydrolyzed with 12N HCl at 120° C., then were mixed with reaction reagents and incubated following the instruction. The final concentration of the hydroxyproline was calculated using the OD reading at 560 nm against the standard curve.

Hydroxyproline quantification is the most common method for the evaluation of tissue collagen deposition. In this study, the results of the hydroxyproline tests showed, while both chemical-treated and untreated macrophages reduced the collagen contents in silica-induced mouse lung tissues, the chemical-treated macrophages group had a static advantage in the fibrosis reduction in comparison to the untreated cells. And this phenomenon occurred in both tail vein delivery mice and the direct intratracheal delivery mice.

### Example 3: Treatment of Fibrosis with Chemically-Treated Macrophages

The following is a prophetic example of using chemically-treated macrophages to treat fibrosis in human patients.

### 1. Fibrosis

The fibrotic disease is a progressive disease characterized by an increase in fibrotic connective tissue and a decrease in normal cells in tissues and organs, which is characterized by difficulties in early diagnosis, complex pathogenesis and poor prognosis. Injured tissues usually heal by secreting collagen, but in cases of severe or repeated injury and dysregulated wound healing response, extracellular matrix (ECM) components, including collagen and fibronectin, accumulate excessively in or around the inflamed or injured tissues and form permanent scars on the tissues and organs, Ultimately, this can lead to organ deformation and functional failure. Injuries associated with a variety of organ diseases can trigger complex cellular and molecular cascades that lead to the development of fibrotic disease.

There are four main stages of organ fibrogenesis, the first of which is the induction of the fibrotic process by primary organ injury, the second is the activation of effector cells associated with the fibrotic process, the third is the subtle alteration of the extracellular matrix, and the fourth is the dynamic deposition of the extracellular matrix. The irreversible progression of the four stages continues to advance the fibrotic process, eventually leading to structural destruction and functional decompensation of the organ and even organ failure. Currently, there are limited therapeutic tools available for fibrotic diseases.

### II. Cell Product Preparation.

The macrophages used in some embodiments described in the present invention can come from a source of peripheral blood mononuclear cells (PBMCs). PBMCs can be isolated from a patient's peripheral blood by gradient centrifuge in Ficoll tubes. After the PBMC are selected from leukapheresis or from a whole blood sample, a human Classical Monocyte Isolation Kit (Miltenyi Biotec, CA) can be used to select CD14+ monocytes, then the CD14+ cells will be cultured in a TexMACs medium (Miltenyi Biotec, CA) supported with 100 ng/ml human M-CSF. On day six of the culturing process, the cells can be treated with MRCi for two hours and then placed under hypoxia condition with fresh medium, and continuously cultured for one more day. On day seven, chemically-treated macrophages will be collected and cryopreserved with cGMP-grade DMSO then the autologous product will be stored in a cGMP-grade vial for shipping to the hospital.

### III. Idiopathic Pulmonary Fibrosis (IPF)

Idiopathic pulmonary fibrosis (IPF) is a devastating interstitial lung disease (ILD) characterized by interstitial scarring of the lung, ultimately leading to parenchymal destruction and loss of normal respirational function. It primarily affects men aged 60-75 years old. The average survival after diagnosis of idiopathic pulmonary fibrosis is 2.8 years, and the 5-year survival is even lower than that of patients with some tumors such as lung and pancreatic cancer, at only 30%. Idiopathic pulmonary fibrosis is therefore a neoplastic disease, even more life-threatening than some tumors. It has been shown that repeated exposure to unknown injurious stimuli leads to abnormal alveolar epithelial function, excessive wound healing response, myofibroblast activation, and deposition of excess extracellular matrix (ECM). Recent studies have highlighted the role of alveolar epithelial cells in disease pathogenesis, and as a result, IPF is considered a multifactorial disease characterized by alveolar epithelial injury and alveolar collapse, alveolar epithelial type II cytopenia, and alveolar stem cell failure. Smoking, viral infections and environmental pollution on genetic susceptibility are considered as potential causative factors.

In some embodiments of this disclosure, chemically-treated autologous macrophages can be used to treat patients with IPF. PBMCs will be isolated from the peripheral blood of patients subjected to the treatment. Macrophages will be differentiated and treated with MRCi following the methods described in the previous section. Frozen autologous cell product then will be thawed and then administered into the patient via intravenous injection.

### IV. Liver Fibrosis and Cirrhosis

Liver fibrosis is a chronic lesion occurring in the liver organ, with the main pathological manifestation being the excessive accumulation of extracellular matrix proteins, including collagen, which in turn is accompanied by the destruction of normal liver tissue structure. Most chronic liver injuries cause fibrotic changes in the liver, including viral infections (hepatitis A, B, and C), alcoholic liver injury, and nonalcoholic steatohepatitis (NASH), among others. If liver fibrosis is not treated in a timely manner, it can progress to cirrhosis, leading to intrahepatic cellular dysfunction and obstruction of blood flow, with the secondary effects of liver failure and portal hypertension posing a direct threat to the patient's life.

There is a lack of clinical tools and drugs that can effectively treat liver fibrosis and cirrhosis. Some non-specific treatments targeting the causative factors, including anti-inflammatory, antiviral, and lifestyle changes, can alleviate the progression of fibrosis to some extent, but so far hepatic fibrosis is still considered by most people as an “irreversible” lesion, and liver transplantation is the only treatment that can fundamentally address liver fibrosis and cirrhosis.

In some embodiments of this disclosure, chemically-treated autologous macrophages can be used to treat patients with liver fibrosis or cirrhosis. Patients subjected to the treatment will first provide PBMCs. Macrophage products will then be developed according to the protocol described in the previous section. After the MRCi-treated macrophages are developed, frozen cells can be thawed and then injected into patients via intravenous or through the portal vein using a fiberoptic device.

### V. Myocardial Fibrosis

Heart disease is the major cause of mortality in developed countries, accounting for an annual death of about 800,000 in the United States alone. Numerous forms of the cardiovascular disease exist that have differential pathological observations. Most cardiac diseases are associated with cardiac fibrosis which refers to an abnormal scarring process of heart valves caused by an inappropriate proliferation of myofibroblast and excessive deposition of extracellular matrix (ECM) proteins in cardiac muscle. Myofibroblasts are principally responsible for the deposition of the excessive fibrotic ECM. The activation of cardiac fibrosis has been extensively studied in the past few decades. In response to acute cardiac injuries like ischemia or myocardium infarction, or chronic disease like hypertension, diabetic cardiomyopathy, Cardiac fibroblast (CFs) within the connective tissue in the heart is activated and transformed to myofibroblasts, which induce excessive extracellular matrix (ECM) deposition.

There are two most common types of cardiac fibrosis, Reactive Interstitial Fibrosis (RIF) and Replacement Fibrosis (RF). RIF is often induced by one or multiple progressive chronic courses (e.g., diabetics and hypertension) that are characterized by diffused deposition of collagen protein (a type of ECM) and increased interstitial compartment volume. RF occurs after acute injury while the expansion of ECM and elevated collagen I deposition replace the dead cardiomyocyte in order to prevent the infarcted myocardium from rupturing. In general, increased cardiac fibrosis leads to distorted organ architecture and function that results in heart failure. During the pathological process of cardiac fibrosis, the necrotic and apoptotic cardiomyocytes trigger the excessive accumulation of ECM proteins in both RIF and RF.

In some embodiments of this disclosure, patients with myocardial fibrosis, either with the subtype of RIF and RF, can be treated with chemically-treated autologous macrophages. First the patients to receive the treatment will provide PBMC as the source of chemically-treated macrophages. The cellular therapeutic product will then subsequently be developed as described in the previous section. For patients with RF type of myocardial fibrosis, the macrophage product can then be administrated into the myocardium around the borderline of the fibrosis site via a fiberoptic device. For patients with RIF type of myocardial fibrosis, cells can be administrated via intravenous injection.

### VI. Renal Fibrosis and Chronic Kidney Disease (CKD)

Similar to the process of fibrotic lesions in other organs, renal fibrosis is characterized by abnormal deposition of extracellular matrix (ECM). As a pathophysiological alteration, renal fibrosis is a progressive process in which the function of the kidney progresses from healthy to injured to damaged until the function is lost. The kidney is stimulated by a variety of pathogenic factors such as trauma, infection, inflammation, blood circulation disorders, and immune response, and its intrinsic cells are damaged, which develop to a large amount of collagen deposition and accumulation at a later stage, resulting in gradual sclerosis and scarring of the renal parenchyma until the kidney completely loses its organ function. The process of fibrosis and sclerosis of the intrinsic cells in the kidney is also the process of renal fibrosis.

Renal fibrosis is also a common manifestation and hallmark of many types of chronic kidney disease (CKD), often appearing in different morphological patterns. In some cases, patients have extensive scarring of the kidney visible to the naked eye, a category often triggered by severe focal injury and complete parenchymal destruction. In addition, chronic glomerular injury leading to corresponding tubular atrophy and degeneration of specific renal units can also eventually lead to interstitial fibrosis/tubular atrophy (IF/TA). In contrast, diffuse fibrosis unrelated to tubular atrophy seems to be a different pathogenic process compared to focal replacement scarring caused by glomeruli. Renal fibrosis appears to develop in a compartment-specific manner, but whether focal and diffuse fibrosis has distinct features associated with other glomerular or tubulointerstitial lesions remains elusive. Patients with chronic kidney disease and secondary renal fibrosis progress relatively slowly, but without timely and targeted diagnosis and treatment, the disease is prone to deteriorate and develop into chronic renal insufficiency, which may eventually lead to end-stage renal disease or even uremia.

In some embodiments of this disclosure, chemically-treated autologous macrophages can be used to treat patients with renal fibrosis or CKD. PBMC will be isolated from patients to receive the cellular therapy, then the PBMC will be used as the source to develop the macrophages as described previously. Once the cells are ready for clinical treatment, they can be administrated either through intravenous injection or percutaneous renal fibroscopic injection locally into the kidney.

Embodiments described herein are directed to use a temperature-responsive cell culture surface for growing and harvesting macrophages for cell therapeutic purposes. More specifically, the said material is the poly (N-isopropylacrylamide) (PIPAAm). Macrophages culture on PIPAAm coated surface grow to confluency at normal cell culture conditions, which is at 37° C. supplied with 5% carbon dioxide. When the culture process reaches to the harvesting points, by reducing the temperature of the culture environment to 20 to 25° C., the macrophages detach from the TCPS without using conventional enzyme-based proteolysis agents like trypsin-EDTA.

The Poly(N-isoproplyacrylamide) (PIPAAm) is a polymer that was first synthesized in the 1950s. It has an intellectual character of structure alteration in response to temperature changes and has been applied to many aspects of cellular biology practices, including tissue engineering and controlled medicine delivery. When the ambient temperature changes, PIPAAm goes through a reversible phase transition between hydrophobic and hydrophilic around a lower critical solution temperature (LCST) of 32° C.

Under normal cell culture conditions with a temperature of 37° C., PIPAAm is hydrophobic, and the polymer forms as collapsed globules, allowing the attachment and proliferation of macrophages similar to those of normal culture dishes. Once environmental temperature reaches below the LCST, like around room temperature, PIPAAm becomes hydrophilic and soluble, and the polymer turns to an extended coil-like formation. Once the hydrophobic to hydrophilic transition occurs, macrophages detach spontaneously and float up freely, without the application of proteolytic enzymes or chelating agents. Traditional cell harvesting methods using trypsin or chelating agents often impair cell surface proteins and the extracellular matrix by cleaving various membrane-associated proteins, which subsequently reduces cell viability and causes unintended characteristic damage to the cell. Culturing macrophages on a PIPAAm-coated TCPS avoids the disadvantages of the traditional method and preserves the cell integrity to a maximum degree.

Applied in the present invention are macrophage progenitors or precursor cells that have been fully differentiated from source macrophages, such as monocytes. These cells are mature macrophages but have not yet been polarized. In some embodiments of the present invention, unpolarized macrophages can be obtained from, for example, CD14-positive monocytes from peripheral blood mononuclear cell (PBMC), followed by isolation and culture of monocytes with supplements of Macrophage colony-stimulating factor (M-CSF) on a TCPS with PIPAAm coating.

In some embodiments, unpolarized macrophages are generated from CD14+ monocytes isolated from human PBMC using a CliniMACS CD14 isolation system (Miltenyi Biotec B.V. & Co. KG, Germany) according to the culture methods and conditions described in the example 4 herein. CD14+ monocytes then can be cultured in a TexMACS medium (Miltenyi Biotec B.V. & Co. KG, Germany) supplied with Macrophage Colony-Stimulating Factor (M-CSF, R&D Systems, MN) at a concentration of 100 ng/ml. Cells can be cultured in either Nunc™ Dishes with UpCell™ Surface (Thermo Fisher Scientific, Waltham, MA) with PIPAAm-coating or regular cell culture dishes from Corning (Tewksbury, MA) as a control. By the end of the culturing process, cells can be harvested via different methods and the cell number and viability can be compared between groups to illustrate the advantage of the PIPAAm-coated dish in culturing macrophages.

The present invention demonstrates the PIPAAm-coated tissue culture vehicles have distinct advantages in the growing and culturing of macrophages in terms of increased cell number and preserving cell integrity, both of which are essential aspects of manufacturing high-quality cell products for future clinical applications.

### Example 4: Culturing Macrophages on PIPAAm-Coated Surface Increases Cell Number and Viability

### I. CD14+ Monocytes Isolation and Differentiation of Macrophages.

In this example, macrophages were isolated and cultivated from the source of fresh human PBMC. Fresh whole blood was diluted with Hank's IX Balanced Salt Solutions (HBSS) (Marlborough, MA) containing 2 mM EDTA (Thermo Fisher Scientific, Waltham, MA) and transferred into LeucoSep™ tubes (Greiner Bio-One International GmbH, Monroe, NC) for separation. After centrifuging at 800 g for 15 minutes, PBMC was harvested from the second layer from the top. Cells were then raised with HBSS/EDTA three times. Cells were then treated with Red Blood Cell Lysis Buffer (MilliporeSigma, MA, USA) by incubation at room temperature for five minutes. After the treatment, cells were washed with HBSS/EDTA and ready for subsequent CD14+ cell isolation.

An isolation buffer was prepared for use with the CliniMACS CD14 Isolation System (Miltenyi Biotec B.V. & Co. KG, Germany). The buffer comprises Phosphate Buffered Saline, 0.1% endotoxin-free Bovine Serum Albumin, and 2 mM EDTA (all from MilliporeSigma, MA, USA). Cell pellets were resuspended with isolation buffer and mixed gently with CliniMACS CD14+ bead for 15 minutes at 4° C. LS Columns from the CliniMACS CD14 Isolation System were placed on a magnetic rack and rinsed with isolation buffer before the cell/beads mixtures were loaded onto the column following three times of washes with the isolation buffer. Upon the completion of washes, cells were collected for subsequent procedures.

CD14+ cells were then divided into three groups, one group was cultured in Nunc™ Dishes with UpCell™ Surface (Thermo Fisher Scientifics, Waltham, MA), the other two groups were cultured in regular tissue culture dishes from Corning (Tewksbury, MA) and will be subjected to different harvesting methods. All groups were cultured in TexMACS medium (Miltenyi Biotec B.V. & Co. KG, Germany) supplied with 100 ng/ml M-CSF Protein (R&D Systems, MN, USA) for seven days at 37° C. with 5% carbon dioxide.

### II. Macrophage Harvesting and Cell Viability Measurement.

To evaluate the advantage of the PIPAAm-coated TCPS in harvesting macrophages, by the end of the seven-day culture process, three groups of cells were subsequently subjected for different harvesting methods. Cells from all groups were removed from the incubator.

Macrophages cultured in the UpCell dishes were placed at room temperature for 25 minutes, then the culture medium containing detached cells were collected into 50 ml centrifuge tubes.

For one group of macrophages cultured in regular tissue culture dishes, culture medium was removed and replaced with ice-cold PBS. Cells were then placed at 4° C. for 10 minutes. At the end of the 10-minute incubation time, cells were collected with Fisherbrand Cell Lifters (Thermo Fisher Scientific, Waltham, MA) by gently scraping off the cells from the dish surface and transferring them into 50 ml centrifuge tubes.

For the other group of macrophages cultured in regular tissue culture dishes, a Gibco Cell Dissociation Buffer was used for harvesting the cell. The buffer is a membrane-filtered, isotonic, and enzyme-free solution of salts, chelating agents, and it claims to be suitable for the dissociation of mammalian cells while preserving cell surface proteins. The culture medium was removed and washed with PBS, then the cells were incubated with the Cell Dissociation Buffer for 10 minutes at 37° C. After the incubation, the buffer containing cells were collected in a 50 ml tube.

Cell number and viability from all three groups were measured using a Cellometer Auto T4 Bright Field Cell Counter (Nexcelom Bioscience, Lawrence, MA).

### Example 5: PBMC-Derived Macrophage Isolation and Treatment

In this example, CD14-positive cells were isolated using the Classical Monocyte Isolation Kits (Miltenyi Biotec Bergisch Gladbach, Germany) from fresh human PBMC. The cells were then cultured for seven days in the presence of 100 ng/ml human M-CSF (Miltenyi Biotec Bergisch Gladbach, Germany) On day seven, the culture media were added with 1 μm Rotenone (Sigma-Aldrich, MO, USA) and the cells were cultured for two hours. By the end of the two-hour incubation time, culture media were replaced with Rotenone-free fresh media which was then placed in a hypoxia chamber with 1% oxygen and cultured for different hours as indicated in the animal treatment section. The cells were then collected and injected via tail vein into BALB/c nude mice with bleomycin-induced IPF.

### 1. Animal Treatments (3, 6 and 12-Hour Hypo)

6-week-old BALB/c nude mice were randomly assigned to one of five groups: Group 1 received intratracheal instillation of 4 U/kg bleomycin only, Group 2 received 4U/kg bleomycin and intravenous injection of PBMC-derived macrophages, and Groups 3, 4 and 5 received 4U/kg bleomycin and intravenous injection of PBMC-derived macrophages that were treated with Rotenone (1 μm) and cultured under hypoxia (1% oxygen) condition for 3, 6 or 12 hours prior to injection respectively. Two weeks after bleomycin instillation, mice in Group 2, 3, 4 and 5 were injected with PBMC-derived macrophages, with or without Rotenone/hypoxia treatments, via tail vein. Mice in Group 1 were injected with PBS via tail vein as a control.

### II. Hydroxyproline Measurements.

One week after macrophage engraftment, mice were sacrificed and the lung hydroxyproline contents were measured using a hydroxyproline kit (Nanjing Jiancheng Bioengineering Institute, Nanjing, China).

### III. Result.

Treatment with PBMC-derived macrophages led to a significant reduction in lung hydroxyproline content, which is an indicator of the extent of lung fibrosis. Compared to the bleomycin+PBS group, all groups that received macrophage treatment showed a significant reduction in hydroxyproline content. The group that received macrophages treated with hypoxia and rotenone showed a larger reduction in hydroxyproline content, with 12-hour hypoxia treatment bringing the most significant reduction, suggesting that this treatment may be particularly effective in treating lung fibrosis. These results suggest that PBMC-derived macrophage treatment has the potential to effectively reduce lung fibrosis induced by bleomycin in mice, with the hypoxia and rotenone treatment showing particular promise.

What is claimed is:

1. A method for treating fibrosis in a subject, comprising administering to the subject macrophages that have been treated with a mitochondrial respiratory chain inhibitor (MRCi) and cultured under hypoxia conditions for a period of time, wherein the MRCi is selected from the group consisting of rotenone, capsaicin, and Piericidin A, and wherein the treated macrophages exhibit enhanced anti-fibrosis ability.

2. The method of claim 1, wherein the macrophages are either bone marrow-derived macrophages, or fresh human PBMC.

3. The method of claim 1, wherein the fibrosis is pulmonary fibrosis, liver fibrosis, myocardial fibrosis or renal fibrosis.

4. The method of claim 1, wherein the macrophages are administered to the subject via tail vein injection, intravenous injection, portal vein injection, intracardiac injection or intratracheal delivery.

5. The method of claim 1, wherein the hypoxia condition includes a low oxygen environment.

6. The method of claim 1, wherein the treated subject exhibits a reduction in hydroxyproline content in the fibrotic tissue when tested with a hydroxyproline colorimetric assay kit.

7. The method of claim 1, wherein the macrophages have been treated with rotenone and cultured under hypoxia conditions for 12 hours.

8. A method of producing macrophages with enhanced anti-fibrotic activity, comprising the steps of:
(a) Treating a sample of macrophages in a culture medium with a mitochondrial respiratory chain inhibitor (MRCi),
(b) Culturing the treated sample of macrophages under hypoxia condition for a period of time, and
(c) Harvesting macrophages with enhanced anti-fibrotic activity.

9. The method of claim 8, wherein the macrophages are either bone marrow-derived macrophages, or fresh human PBMC.

10. The method of claim 8, wherein the MRCi is rotenone, capsaicin, or Piericidin A.

11. The method of claim 8, wherein the treated sample of macrophages is cultured under hypoxia condition for up to 12 hours.

12. The method according to claim 8, further comprising a method for enhancing cell yields and viability, wherein cells are cultured in UpCell dishes coated with PIPAAm.

13. The method of claim 12, wherein the cells are harvested in higher yield and with higher viability in compared to cells cultured in traditional methods.

14. A macrophage with enhanced anti-fibrotic activity made by a method according to claim 8.

15. A macrophage with enhanced anti-fibrotic activity made by a method according to claim 9.

16. A macrophage with enhanced anti-fibrotic activity made by a method according to claim 10.

17. A macrophage with enhanced anti-fibrotic activity made by a method according to claim 11.

18. A macrophage with enhanced anti-fibrotic activity made by a method according to claim 12.

19. A macrophage with enhanced anti-fibrotic activity made by a method according to claim 13.
